(19) United States
(12) Patent Application Publication
(10) Pub. No.: US 2008/0125757 A1
(43) Pub. Date: May 29, 2008

(54) Indwelling urinary catheter with enlarged sealing surface

(76) Inventor: Fred Gobel, Wilhelmsfeld (DE)

Correspondence Address: KIMBERLY-CLARK WORLDWIDE, INC.;Catherine E. Wolf, 401 NORTH LAKE STREET, NEENAH, WI, 54956, US

(21) Appl. No.: 11/526,442

(22) Filed: Sep. 25, 2006

(51) Int. Cl. A61M 27/00

(52) U.S. Cl. 604/544

(57) ABSTRACT

An indwelling urinary catheter for transurethral introduction into a urinary bladder is shown and described herein. The catheter includes a flexible catheter shaft having a wall, a filling channel is integrated into the wall, and a thin foil balloon element having a film-like structure. The balloon element when positioned in the bladder is adapted to contact internal urinary bladder structures over at least about 30 percent of a balloon element surface area. The seal is greater than seals of equivalent balloon structures.

### BACKGROUND OF THE INVENTION

The present invention is directed to a urinary catheter for transurethral introduction into the urinary bladder through the urethra. More particularly, the present invention is directed to an indwelling urinary catheter that conforms more readily to the trigonum vesicae structure of the bladder thus better sealing the internal urethral orifice and reducing the incidence of ulceration of the bladder structures.

When providing health care, the use of a urinary catheter is often required. The indwelling urinary catheter most often in use today is the Foley catheter. A Foley catheter is composed of a flexible catheter shaft having a distal end which is placed in the patient's bladder. A fluid-refillable balloon element is fastened to the shaft. The catheter shaft has a filling channel, which leads into the balloon interior via an opening in the catheter wall. The main purpose of the balloon element is to securely mechanically anchor the catheter in the urinary bladder. In addition, when placed proximate the internal urethral orifice or exit from the bladder, the balloon has a certain sealing function which is intended to prevent urine from flowing out of the bladder, past the catheter and through the urethra.

In the unfilled state, the balloon element resembles a sleeve pulled over the catheter shaft, fitting 360 degrees around the shaft, typically under slight tensioning, in any case, however, in a fold-free manner. The sleeve is comparable to a hose fitting, and is usually fabricated from the same material or a substantially identical material as the shaft, but is modified in its elongation properties.

Conventional balloon elements are designed with this specific type of construction, which, in the emptied state, fit closely on the shaft to enable the balloon element to be advanced with as little resistance as possible through the urethra and into the bladder lumen. In this way, painful irritations or lesions of the urethra's mucous membrane, caused by folds or bulges in the wall of the balloon element that previously existed or formed during the advancing motion, are avoided when inserting the catheter.

In previous embodiments it was already known to manufacture a polyurethane shaft using an extrusion method for indwelling urinary catheters. This method has been tried and tested in clinical applications on patients. Unfortunately, due to its inadequate elongation properties, polyurethane has long been considered unsuitable for balloon elements of conventional design types. This is because the balloon, once securely introduced into the bladder is required to be elastically expanded into a balloon by injection of a fluid, under relatively high pressure while remaining closely fitted on the shaft at the regions where it is secured thereto.

For that reason, catheters having a polyurethane shaft were provided in known methods heretofore with balloon elements of latex or silicon or of related, similarly volume-expandable materials. The material normally selected for the catheter shalt and the balloon element of conventional catheters typically contains latex or silicon, and as such permits an elastic expansion of the balloon element to a volume of 5 and 30 ml, respectively. These are the two standard balloon volumes for indwelling urinary catheters used in clinical practice. As such, a polyurethane sleeve that is pulled over the shaft (typical shaft diameter of approximately 4 to 6 mm for adults) and fits closely thereon, could only be elastically expanded to a balloon of a sufficient size (filling volume 5 or 30 ml) under very high pressure, which was only able to be conditionally produced by the user using conventional means. The stresses produced in the wall of the balloon being shaped out would be considerable. As a result any drainage lumen of the catheter would be substantially constricted by the immense balloon filling pressure.

Moreover, ideally, the sleeve, that has been elastically expanded into a balloon, fully retracts, even after a longer-term use of the catheter, and will again closely fit on the catheter shaft as a sleeve-type hose fitting, without forming folds or bulges. In tis way, the drained balloon does not cause any painful irritation or trauma to the sensitive urethra epithelium even during removal of the catheter. Typically, however, the sleeve that has been elastically expanded for an extended period of time into a balloon, is not able to be fully elastically retracted onto the shaft. The partial loss of the sleeve elasticity caused by an elastic expansion of the balloon over several days can be accelerated by the action of chemically aggressive urinary components (e.g., uric acid). In the case of latex-based catheters, given a long-period use, the urine regularly leads to a pronounced stiffening of the balloon, but also to a considerable loss of elasticity of the catheter shaft itself. Once drained, balloons of the known type of construction, having a latex- or silicon-based sleeve, often exhibit residual, coarse folds or bulges in the (not fully) retracting sleeve. This situation often poses a considerable risk of injury to the patient.

Moreover, catheter materials customarily used up to now (latex, silicon, or latex- or silicon-based materials, and/or composite materials made of latex and silicon) have other clinically relevant disadvantages.

One drawback (particularly when latex materials are used) is that the balloon element does not always inflate symmetrically when elastically expanding and may burst in response to any slight lateral weighting. The stability of the balloon anchoring in the opening of the bladder may also be adversely affected by a pronounced asymmetry of the balloon with respect to its shape. Moreover, a pronounced asymmetry of the filled balloon element, depending on its placement in the opening of the bladder, may in some instances cause the catheter lumen to snap off. Latex has also been associated with allergic responses in some individuals and as such its use is becoming less desirable in the medical field.

A final disadvantage is that the balloon element of catheters of a conventional type of construction, as necessitated by the particularities of the manufacturing and the material, must remain below specific wall thicknesses. The minimum wall thickness of the elastically expanding sleeve, when filled to form the balloon, must be such that it is able to avoid, with certainty, falling below a lower, critical minimum wall thickness, below which the danger of rupture exists, in response to increasing shaping-out of the balloon (and the reduction in the balloon wall thickness accompanying the elastic expansion).

The minimum wall thickness of the balloon element that fits on the shaft in the manner of a sleeve is typically within the range of at least 100 micrometers and requires relatively high pressures when the sleeve undergoes elastic expansion or deformation. During expansion, the balloon element assumes a shape predominantly in the radial, but also in the longitudinal direction (elongation). With increasing filling volume, the pressures forming in response to the predominantly radial elastic expansion of the balloon envelope in many cases cause a compression or stenosis of the drainage lumen of the catheter. This lumen-narrowing effect is further complicated by the elastic expansion of the balloon in the longitudinal direction and, as a consequence thereof, the elongation of the catheter shaft in the balloon region. Both elongation components may lead to a considerable narrowing or stenosis of the catheter lumen.

It is a complex process to manufacture conventional indwelling urinary catheters, and one that requires many individual steps. As such, many other issues arise which may affect the catheter's suitability to the purpose intended. In many cases, the particular dipping or molding processes do not ensure a satisfactory surface quality of the catheter and balloon. Silicon processing yields slightly rough and irregular boundary surfaces which promotes the encrustation of urinary components, as well as bacterial colonization of catheter surfaces. Another issue of particular difficulty associated with silicon is that of water substantially permeating through the balloon envelope. To ensure that the balloon is adequately filled it must typically be refilled in an almost daily cycle.

### SUMMARY OF THE INVENTION

As such in one aspect, the present invention serves to avoid the above-mentioned disadvantages associated with the catheters described above and to devise an indwelling urinary catheter capable of simple manufacture from a standpoint of production engineering. Such a catheter should be designed to expand the existing art by providing a special embodiment of a urinary catheter, specifically designed for the requirements of longterm catheterization and the prevention of complications associated with longterm placement of a balloon-equipped catheter in the urinary bladder. Use of the device is anticipated to reduce the incidence of pressure induced ulcers due to the continuous mechanical irritation of bladder tissue typically associated with prior art high pressured balloons, wherein a stiff, inflexible, fully distended balloon structure is made to rest within the trigonum-shaped outlet-portion of the urinary bladder, causing typical force-induced trauma at the resting area between balloon and bladder.

Unlike in the prior art balloons, the balloon of the present device is preformed to a defined fully inflated volume prior to affixing it to the catheter shaft. Once attached to the shaft, it may be placed in a first condition, that is, a resting volume or base state, characterized in that the balloon is deflated and collapsed into a film-like structure. The collapsed film-like structure does not meaningfully increase the outer diameter of the shaft and as such the shaft and collapsed balloon are insertable through the urethra and removable without increasing urethral irritation or trauma.

Once the catheter is positioned, the balloon is inflated to a working volume which is less than that of its fully inflated volume. At this working volume, the balloon would better conform to the shape of the bladder and more specifically the trigonum vesicae. Due to the balloon being inflated to a volume less than its capacity, the balloon foil itself could be made to contact more of the bladder wall. This would increase the sealing properties of the catheter further minimizing leakage of urine past the balloon as well as the ascension of bacteria from the external environment, up through the urethra, and into the bladder. Moreover the balloon may be coated so as to have an antimicrobial effect as well as to minimize the encrustation of urine based compounds on the outside as well as the inside surfaces of the balloon.

One such catheter described in accordance with the present invention would be simple to manufacture in terms of production engineering and would eliminate the need for cost-intensive manufacturing steps in comparison to conventional catheter types, such as, above all, latex catheters which are manufactured using a dipping method. Additionally, the described balloon further overcomes the problem of changing material mechanics, specifically, the stiffening of the material after prolonged inflation which is associated with conventional materials often causing traumatic irritation in long-term catheterization.

In one embodiment, an indwelling urinary catheter for transurethral introduction into a urinary bladder is provided. The device has a flexible catheter shaft having a wall, a filling channel integrated into the wall, and a thin foil balloon element having a film-like structure that when positioned in the bladder is adapted to contact internal urinary bladder structures over a substantial portion of the balloon element surface area. In some embodiments, the contact area is between about 30 to about 60 percent of the balloon element surface area.

In another embodiment of the present invention, a method is disclosed. The method includes the steps of introducing a flexible catheter having a balloon through the urethra and into the bladder. Once placed the user may inflate the balloon in a range of between about 50 to about 80 percent of its pre-shaped or fully inflated volume. The sealing effect of the balloon may be increased by subjecting the catheter to a tractive force while it is intravesically in situ. In embodiments similar to tis, the balloon may contact the internal urinary bladder structures over a range from at least about 30 percent of the balloon surface area to at least about 60 percent of the balloon surface area.

### BRIEF DESCRIPTION OF THE DRAWINGS

The present invention is explained in greater detail on the basis of exemplary embodiments illustrated in the drawings, wherein:

FIG. 1 is a lateral view of the distal end of one embodiment of catheter in accordance with the present invention prior to its insertion into a urinary bladder; and

FIG. 2 is a lateral part sectional view of tie catheter of the FIG. 1 catheter while it is intravesically in situ.

### DETAILED DESCRIPTION OF THE PRESENT INVENTION

In response to the foregoing challenges that have been experienced by those of skill in the art, the present invention is directed toward an improved indwelling urinary catheter that conforms more readily to the trigonum vesicae structure of the bladder thus better sealing the internal urethral orifice and reducing the incidence of ulceration of the bladder structures and infection of the same from transurethral migration of bacterium.

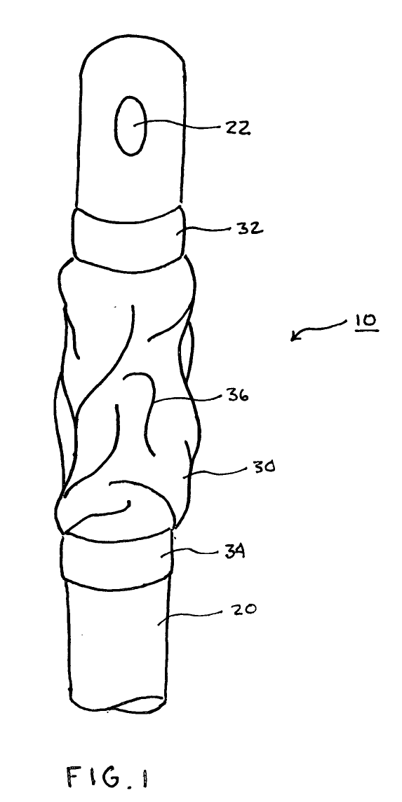

FIG. 1 shows the distal end of an indwelling urinary catheter 10 in a lateral illustrative view. The catheter 10 is provided with a shaft 20 and a balloon 30 affixed to the shaft 20. Proximal to the distal end of the shaft 20 is an orifice 22 which serves to drain urine or other fluids through the interior of the catheter 10. The balloon 30 is shown in a base-state, i.e. at rest and fully collapsed. Bands 32 and 34 are formed by the ends of the balloon 30 and serve as fluid tight bonds between the shaft 20 and the balloon 30. The bonding of the bands themselves may be formed by a suitable adhesive, ultrasonic welding, or other bonding technique known and understood by those of skill in the art.

Surprisingly in contrast to the above description, it turns out, that a polyurethane balloon may nevertheless be used when manufacturing an indwelling urinary catheter, particularly when the balloon is preformed into a balloon film, foil, or sleeve having a wall thickness of about 3 to about 20 micrometers, In many embodiments, this sleeve may often fall between the ranges of from about 5 to about 15 micrometers in thickness. The sleeve is fitted closely on the shaft wall in an emptied or collapsed state, the film-like structure folding in randomly or in preconfigured patterns. The sleeve is provided with two shaft attachment pieces which are fastened to the catheter shalt 20, i.e., the bands 32 and 34 respectively. As such, with this embodiment, there is no need to reduce the shaft diameter so, the balloon 30 sits or lays folded on the shaft 20. This enables the user to select the shaft thickness of the catheter in the usual manner without any restrictions based upon the balloon increasing the diameter of the shaft in any significant manner.

It has been found that according to some embodiments the material selected to form the sleeve or balloon 30 may include materials of low compliance such as polyurethane (PU), low-density polyethylene (LDPE), polyvinyl chloride (PVC), polyamid (PA) or polyethylene teraphthalate (PETP). These materials are biocompatible and, when being processed into correspondingly thin walls, are especially suited for forming the balloon. Copolymer admixtures for modifying the characteristics of the material are also possible, for example a low density polyethylene and ethylene-vinylacetate copolymer (LDPE-EVA), or blends of the above mentioned materials (e.g. PU with PVC or PU with PA) would be considered suitable for such a use. Other materials would also be suitable so long as they exhibit properties enabling them to be processed into balloons having microthin walls on the order of about 3 to about 20 micrometers, many embodiments falling into the range of between about 5 to about 15 micrometers. Suitable materials should possess properties enabling them to be processed into anchor mechanisms having microthin walls which do not deform elastically to such a degree that they are enabled to slip through the insertion channel in the body wall, in this case, the urethra.

Some of the characteristics of the device may include but are not limited to: a soft pliable foil material, which can be reduced in thickness down to the low micrometer range, from about 3 to about 20 microns; a material, when blow-molded, dip-molded or welded from foils at the above thicknesses, grants compliance characteristics securing the shaping-of a retention body, not breaking out or bulging in shape to such a degree that the balloon can be luxated into or through the urethra; the balloon shape may be spherical, elliptical, or conical; and the balloon may be dimensioned residually, such that the creation of hull or envelope infolding occurs.

In its collapsed state, the balloon 30 is fitted on the surface of the shaft 20 as shown in FIG. 1 and described above. The balloon 30 is bonded or fused to the catheter shaft 20 at the bands 32 and 34 also as indicated above. In the manufacturing of the base-state balloon, the transition regions from the bands to the central, mid-position diameter of the base-state balloon may be designed to have wall thicknesses which continuously decrease from the bands to the central, mid-position diameter. Moreover, it may be advantageous when, after joining the balloon to the catheter shaft, the end rims of the bands are smoothed, for example, by the action of heat or via the application of solvents, so that no sharp-edged transitions are present in the transition region from the shaft 20 to the balloon 30. The transition regions from the bands 32 and 34 to the central, mid-position section of balloon 30 are also kept as a continuous, fluid transition, so that if the wall thicknesses do vary, they would continuously decrease from the thickness at the bands to the thickness at the central, mid-position diameter of base-state balloon 30.

In addition, the surfaces of both the balloon 30 and, respectively, of the extruded shaft 20, both of which may be fabricated using the blow-molding method are of the highest quality when polyurethane is used. Smooth, high quality surface topography of the components minimizes encrustation by urinary components, as well as minimizes bacterial colonization which is rendered difficult by the resulting surface evenness. As stated, this may be accomplished by appropriate use of the blow-molding process. The process is based on precision extruded raw tubing, which in a second step is taken under stretch and then inflated in a hot molding procedure into the balloon shape. The surface properties achieved are considerably better than with conventional Foley balloon materials, which are characterized by a cratered, uneven surface, which of course facilitates the adhesion and encrustation of urinary components as well as fosters the growth of bacteria.

Furthermore, when polyurethane is used for the catheter shaft 20, the wall thickness of the catheter shaft may also be advantageously reduced than would be found in previous designs, enabling the catheter drainage lumen 22 to be enlarged, given an equivalent external shaft diameter. Thus, given a favorable material selection, a shaft wall thickness of from about 0.4 to about 0.8 mm, and in some embodiments from about 0.4 to about 0.6 mm would suffice. The catheter shaft 20 should nevertheless retain its rigidity or safety against buckling, as required for insertion into the urethra in patient applications.

To further reduce the catheter wall thickness, the catheter shaft 20 may be formed from two concentrically extruded tubes, the inner tube preferably being designed to be thinner and harder than the outer tube. This configuration may be formed by co-extrusion of the tubing, a process known and understood by those having skill in the manufacture of extruded tubing. An alternative means of achieving the same objective may include the use of a reinforcement insert such as a wire, a spiral wound reinforcement, or a stabilizing mesh incorporated into the wall of the shaft.

As may be seen in FIG. 1, and referred to above, the collapsed balloon 30 may contain random patternless fold formations 36. The fold formations 36 may run in any direction, and thus, for example, may also be at transverse or right angles to the shaft axis. Yet, since the foil thickness or balloon wall is exceptionally thin, once the balloon 30 is drained, it would typically cling very closely to the surface of the catheter shaft 20. In some instances, it may even form hanging sack-like folds when the catheter is inserted or removed. It should be realized that due to the wall-thickness ranges contemplated in accordance with the present invention, such hanging sack-like folds would have no disadvantageous or deleterious effect to the patient during passage of the balloon through the urethra. The mechanics of a thin film, low compliance material balloon when mounted to a catheter shaft and deflated, would permit easy insertion and removal of the device from the urethra without causing tissue trauma to the patient.

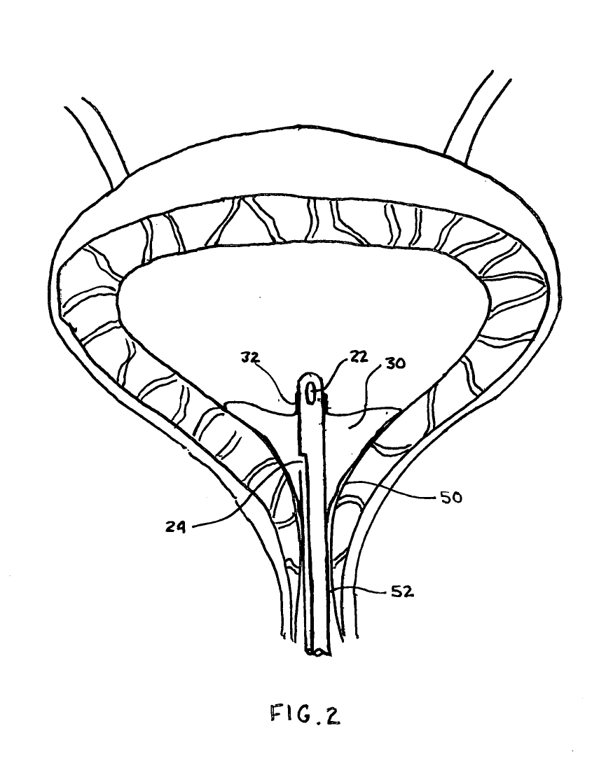

Other embodiments, not shown, may contain fold formations aligned longitudinally with the shaft 20 so as to run substantially between the two bands 32 and 34. In any event, the fold formations permit an expansion of the balloon 30 during inflation, which in turn leads to an in-use, working-state, or working volume configuration as depicted in FIG. 2. Moreover, to reduce the overall space required by any embodiment of fold formation to the greatest degree possible, the balloon may be mounted on tie shaft in the longitudinally oriented form such that the shaft pieces or bands of tie balloon are spaced as far apart as possible without tensioning the balloon envelope.

As may be seen in FIG. 2, the balloon 30 may be inflated sufficiently to partially fill the volume of the balloon yet allow the balloon to remain at ambient pressure, that is, the pressure in the interior of the balloon 30 would equal approximately the pressure found on the balloon's exterior. This condition would enable the configuration of the balloon to shape anatomically into the trigonum vesicae 50 and to fill out the internal urethral orifice 52.

To enable filling of the balloon 30, the catheter shaft 20 is provided with an opening 24, or a plurality of such openings in the region covered by the balloon 30. These filling openings 24 need not be round and actually may have a square or rectangular shape. This shape has been found to substantially prevent the tin film of tie balloon from being able to occlude tie opening or openings.

It should be understood that a partially filled, unpressurized, pre-shaped balloon, made from a material of low compliance, when moved or pulled into the trigonum vesicae would also adjust to the individual's anatomy by enabling the balloon under low force exertion to conform to the anatomical structures which it is resting upon, thereby preventing exertion of force peaks and any resulting ulcers on the prominent bladder structures. This contact is significantly different from the contact associated with the placement of conventional sleeve based, high-pressured balloons which exhibit a ring-like contact area within the bladder. In the present embodiment, the contact area between the balloon and the bladder is larger, since the continuously drained bladder would more or less collapse over the balloon. This structure would also effectively increase the seal of the balloon with internal urethral orifice. In many embodiments of the present invention, at least fully 30 percent of the surface area of the balloon foil or envelope is in contact with the urinary bladder structures of the patient's anatomy. In many embodiments as much as 60 percent of the surface area of the balloon foil or envelope is in contact with the urinary bladder structures.

Due to the evenly distributed surface contact of the balloon along the bladder and trigonum vesicae, the pressure points associated with prior art catheters which cause tissue trauma and foster pressure ulcers are in most instances eliminated or at least reduced. Furthermore, due to the increased contact and sealing area of the present invention within the trigonum, leakage of urine is also reduced past the balloon. Moreover, there should be a commensurate reduction in the ascension of inflammation inducing bacteria into the bladder since there is reduced fluid communication between the urethra and the urinary bladder.

As referred to above, the proposed balloon, in its working state, is designed to be filled with a volume less than that of a similarly sized balloon which is fully inflated, that is, the present balloon is designed to be inflated less than its pre-given shape and dimension would otherwise allow. The balloon hull or envelope therefore is not going to be placed in a state of continuous distension or full inflation, but would be inflated to a volume something less than that. Furthermore, the pressure within the balloon is substantially equal to the pressure at the exterior of the balloon, e.g. the intra-bladder pressure. In some embodiments, the balloon is filled somewhere in the range of between about 50 to about 80 percent of its pre-shaped or fully inflated volume. The balloon may be filled with a liquid or gaseous fluid. The balloon at its working volume state would exhibit a floppy consistency and would not be fully distended, enabling it to fully occupy the trigonum vesicae portion of the urinary bladder, thereby enabling a portion of the balloon to move into the transition portion between bladder trigonum and urethra, or the internal urethral orifice.

When taken under axial, outwardly directed tractive force from the bladder, the balloon would conform to or adapt itself into a conus-like geometry as depicted in FIG. 2, thus anchoring it ever more firmly within the trigonum vesicae 50 and the internal urethral orifice 52. An additional structural advantage to this configuration is that under such a tractive force, the surface of the balloon hull proximate to the orifice 22 and the band 32 would actually form a concavity which would act as a pocket or pool within which urine would collect. In contrast, prior art fully inflated balloon would not be capable of such a configuration and some urine would collect at the confluence of the balloon seating surface with the trigonum vesicae.

An additional feature of the present invention is that due to the limited compliance of the balloon envelope, the balloon is prevented from deforming to such an extent that it would slip inadvertently through and out the urethra. The increase in internal balloon pressure that would occur under conditions of an outward directed axial pulling or tractive force would directly correspond to the axially acting tractive force itself which was placed on the catheter. As such, this enables the intravesical balloon, in its resting state to be maintained at the lowest possible balloon pressure, yet, in situations of demand, the peak pressure it would be forced to withstand would be no greater than the tractive or pull force applied. The overall exertion of force on the bladder structures is therefore limited to the least possible amount and is certainly much less than in prior art devices.

This minimized force-exertion makes the device ideally applicable in post-surgical use, where the catheter balloon has a larger filling volume (up to 80 ml) so as to be used as a space filler within the resection cavity after a prostatic gland removal. In order to effect the best possible fit into the cavity and to exert a certain hemostatic effect, such catheters are usually taken under an active tractive pull force. With the correspondence between tissue exerted force and applied pull force previously described, the externally applied pull force may be reduced sufficiently so as not to interfere with perfusion in the resection cavity wound. This would serve to reduce the negative effect of balloon placement on perfusion.

In any of the embodiments, the dimensional design of the base-state balloon may be calculated, i.e., its wall thickness may be selected in a way that allows the envelope to be elastically expanded up to its working volume, while avoiding a non-elastic overstretching so that the elasticity of the balloon material is completely retained, even in the case of long-term catheter use.

Although various balloon sizes are contemplated, suitable ranges for establishing a working volume may be fashioned by inflating the balloon to within about 60 to about 80 percent of its fully inflated, but not elastically distended state. Other examples may place the balloon at about one-third to about two-thirds full.

Of course other examples may be appropriate in certain instances as well. As one such example, to achieve a working filling volume of 5 ml using filling pressure values that do not compromise the catheter shaft, the base-state balloon is designed in such a way that, in the unexpanded at-rest or base state, i.e., when the balloon is filled to the freely unfolded at-rest or base-state form it has an at-rest or base-state volume of approximately 1.2 to about 2.5 ml. In this filled base state, the cuff envelope would remain unexpanded. Given a larger working filling volume of, for example, 30 ml, in the unexpanded base state, i.e., when filling the balloon to the freely unfolded at-rest form, the base-state balloon would receive a volume at rest of approximately 4 to about 10 ml.

The balloon is preferably fastened to the shaft in a longitudinally extended form as described above. The at-rest volume of the cuff applied in this manner is typically less than 0.08 ml, preferably in the range from only 0.02 to 0.04 ml. In many of the embodiments, the preformed balloon elements may have a working volume of 5 ml and a wall-thickness range of from about 5 to about 10 micrometers. In the case of those specific embodiments having a 30 ml working filling volume, the wall thickness of the balloon envelope may preferably fall within the range of from about 5 to about 15 micrometers.

In the process of manufacturing the catheter, the band portions of the balloon may be fixed to the shaft in such a way that they are maximally spaced apart, while avoiding a tensile stretching of the balloon envelope. This is to enable the balloon envelope to orient itself in a shaft-parallel lengthwise fold formation, so that it clings closely to the catheter shaft. The spacing may be lessened should a random folding pattern be desired. In either embodiment, the remaining at-rest filling volume in tie balloon fastened in this manner, may be made to be typically less than 0.05 ml, and in many embodiments may fall within the range of only about 0.01 to about 0.03 ml.

In those embodiments where polyurethane polymer is used, the uninflated volume of the base-state balloon, and the wall thickness of the balloon foil are selected in such a way that the safety range of volumetric expandability of the balloon falls within a range from about 100 to about 200 percent and does not exceed a safety range of from 200 to about 250 percent. For the balloon according to the present invention, Pellethane 2363 materials having a Shore hardness of 70 to 90 along with their respective subforms (A,AE) may be used in some embodiments of the balloon. As described in more general terms above, materials of other manufacturers having comparable technical material data may be used as well.

To further improve the anti-encrustion performance of the device, a silver particle coating may be applied. The coating could be made to coat the balloon surface, as well as the outside and the inside lumina of the shaft element and may be accomplished during the dipping process. Due to the coating being applied to the balloon when the balloon is freely inflated, when the balloon is placed in its working volume state, that is when it is non-pressurized and non-extended, the silver particles would be in the highest possible density. The migration of the particles apart from one another may be prevented. This is in contrast to conventional sleeve based catheter technology in which the coating is applied to the sleeve in a collapsed or uninflated state, resulting in a considerable degree of such migration in the form of cracking of the coating as the balloon surface expands under inflation, this leaves areas with lower particle or antimicrobial agent density.

The anti-inflammatory effect may also be increased by the silver based coating as well as an antiseptic, antimicrobial coating of various kinds as would be understood by those of skill in the art. A distinction being in their application as disclosed above, i.e., the coating is applied to the inflated balloon and during use the balloon would exhibit uniform coating coverage and efficacy. Additionally the balloon may be filled with a fluid containing an antiseptic or antibiotic solution which when used as a filling medium, would migrate through the balloon membrane due in part to the polar charged molecules of the solution.

Another embodiment of this invention is a urinary catheter, including the balloon, shaft, and any molded valve cavities made completely of polyethylene. While utilizing the same characteristics as mentioned above, this catheter may be manufactured at a fraction of the cost while maintaining many of the improvements enumerated above. Moreover catheters made of polyethylene possess desirable shaft properties including smooth surfaces, soft and flexible physical traits yet they are substantially kink resistant as well. Blow molded, thin membrane polyethylene balloons maintain similar properties but are less expensive to make than other balloons as little to no heat is required during the molding process, serial multiple cavity blowing from one piece of raw tube is possible. Polyethylene raw materials are inexpensive. Since other materials do not bond readily with polyethylene this allows for an inexpensive balloon to be mounted to a shaft with desirable characteristics as described above.

As used herein and in the claims, the term “comprising” is inclusive or open-ended and does not exclude additional unrecited elements, compositional components, or method steps.

While various patents may have been incorporated herein by reference, to the extent there is any inconsistency between incorporated material and that of the written specification, the written specification shall control. In addition, while the invention has been described in detail with respect to specific embodiments thereof, it will be apparent to those skilled in the art that various alterations, modifications and other changes may be made to the invention without departing from the spirit and scope of the present invention. It is therefore intended that the claims cover all such modifications, alterations and other changes encompassed by the appended claims.

1. An indwelling urinary catheter for transurethral introduction into a urinary bladder comprising:
a flexible catheter shaft having a wall;
a filling channel integrated into the wall; and
a thin foil balloon element having a film-like structure that when positioned in the bladder is adapted to contact internal urinary bladder structures over at least about 30 percent of a balloon element surface area.

2. The catheter of claim 1 adapted to reduce trauma where the balloon rests upon internal urinary bladder structures contacting a greater area within tie bladder.

3. The catheter of claim 1 wherein the balloon is inflated and coated with an antimicrobial substance.

4. The catheter of claim 1 wherein the balloon is inflated and coated with an silver containing compound.

5. The catheter of claim 1 comprising polyurethane.

6. The catheter of claim 1 wherein the balloon comprises random patternless folds in an uninflated state.

7. The catheter of claim 1 wherein the balloon forms a concavity proximal to an orifice which serves to drain fluids from a point exterior to the catheter through an interior of the catheter.

8. The catheter of claim 1 wherein the balloon element is adapted to contact internal urinary bladder structures over at least about 60 percent of the balloon element surface area.

9. A method of sealing a urinary bladder comprising:
introducing a flexible catheter having a balloon through the urethra and into the bladder;
inflating the balloon in a range of between about 50 to about 80 percent of its pre-shaped or fully inflated volume.

10. The method of claim 9 comprising increasing the sealing effect of the balloon by subjecting the catheter to a tractive force while it is intravesically in situ.

11. The method of claim 9 comprising contacting the internal urinary bladder structures over at least about 30 percent of the balloon surface area.

12. The method of claim 9 comprising contacting the internal urinary bladder structures over at least about 60 percent of the balloon surface area.
